(19) United States
(12) Patent Application Publication
(10) Pub. No.: US 2005/0229939 A1
(43) Pub. Date: Oct. 20, 2005

(54) Prevention and treatment of disc herniation

(76) Inventor: Anuthep Benja-Athon, New York, NY (US)

Correspondence Address: ANUTHEP BENJA-ATHON, M.D.;Pain Management, Acupuncture Physical Therapy, 210 E 36th St., New York, NY, 10016, US

(21) Appl. No.: 10/825,165

(22) Filed: Apr. 16, 2004

(51) Int. Cl. A61B 19/00

(52) U.S. Cl. 128/898

(57) ABSTRACT

The present invention comprises a method and a data means for preventing and treating spinal disc herniations. The method comprises a discovery that the torques, forces, stresses, strains, sprains imposed on a human spine and pelvis result in the bulging and/or herniation of an intervertebral disc. A formula or a plurality of formulas for the treatments of the soft tissues producing or involving with said disc herniation and a data means for predicting or estimating the risks, incidences and propensity towards said disc herniation in people are provided.

### FIELD OF INVENTION

A method of preventing and nonsurgically treating disc herniation.

### FIELD OF THE INVENTION

The first objective is to prevent spinal disc herniation in humans and animals.

The second objective is to effectively treat and manage spinal disc herniation in humans and animals.

The third objective is to optimize the quality of life and standard of living of humans and animals.

All humans and most animals, inevitably, suffer low back pain and related disorders. Cervical, thoracic and lumbosacral disc herniations are extremely common and the incidences of disc herniation are increasing in U.S.A. Disc herniation and related disorders afflict immense acute and chronic pain, suffering, extreme debilitations and loss of productivities in individuals. Furthermore, said afflictions mostly occurred in individuals in their prime and most productive years of life.

Said disc herniations in said individual are heretofore poorly understood. Consequently, the prevention of said herniations are not available and nonsurgical treatments and managements have been random, ineffective and, in some instances, detrimental. Consequently, exhausted of options, patients having endured pain, suffering and neuromusculoskeletal disorders and deficits were forced to undertake surgery—albeit poor option—of their disc and only to endure and suffer more chronic pain, long-term rehabilitation, changes of life styles, loss of productivities and increased absenteeism. Eventually, many will have repeated low back surgeries and said sequelae.

In summary, the prevention of said disc herniation is heretofore unavailable and all current treatments and managements of said disc herniation are random, ineffective or unnecessary, and, worse, some are detriment to individuals.

Through the research of this physician applicant, the present invention provides a method of and means for effectively preventing and treating intervertebral disc herniation in human and animal spines. Humanity will immensely benefit from the present invention.

### SUMMARY OF THE INVENTION

Spinal disc herniations are extremely common and debilitating in individuals. Said disc herniations are heretofore poorly understood and consequently patients are needlessly subjected to surgery of the discs.

The present invention comprises a method and a data means for preventing and treating spinal disc herniations in humans or animals. The method comprises a discovery that the torques, forces, stresses, strains, sprains imposed on a human spine, pelvis, the spinal and pelvic components result in the bulging and/or herniation of an intervertebral disc or a plurality of intervertebral discs in said spine. The present invention then provides a formula or a plurality of formulas for the treatments of the soft tissues producing or involving with said disc herniation. Furthermore, the present invention provides a data means for predicting or estimating the risks, incidences and propensity towards said disc herniation in said individuals.

### DESCRIPTION OF THE PREFERRED EMBODIMENTS

The present invention comprises a method and a means for preventing and treating cervical, thoracic, lumbosacral intervertebral disc herniations and related disorders in a human or an animal spine.

The method comprises a discovery that the torques, forces, stresses, strains, sprains imposed on a human spine, pelvis, the spinal and pelvic components such as, but not limited to, intervertebral muscles, muscles originating from and inserting on the spine and pelvis, ligaments, intervertebral discs, blood vessels, nerves and joints result in the bulging and/or herniation of an intervertebral disc or a plurality of intervertebral discs in said spine. Moreover, said herniation is not a random process. In other words, there exists a causal relationship between said phenomena, the kinesiology and physiology including the contractility, tensiles, pulls and lengths of said intervertebral muscles and muscles originating from and inserting on the spine, pelvis and legs and said herniation or herniations.

For example, in brief, said disc herniation is a consequence of an imbalance or a poor kinesiologic and biomechanic interactions between a plurality of muscle groups such as, but not limited to, the intervertebral muscles and hip flexors and extensors and the deconditioning, weakness or imbalances of the abdominal and/or back muscles. Similar roles play by the ankle dorsiflexors and ankle dorsiextensors, ligaments or a combination thereof are also implicated.

In other words, the shortening and contracture of a specific muscle or a plurality of muscles relating to said spine and pelvis causes or cause said disc herniation. For example, the shortening, spasm, contracture or a combination thereof of the hip flexors cause said disc herniation. Alternatively, the shortening, spasm, contracture or a combination thereof of the hip extensors cause said disc herniation. In summary, the discovery provides the method of identifying the specific muscle or muscles in the muscle groups of intervertebral muscles, abdominal and back muscles, hip flexors, hip extensors, ankle dorsiflexors and ankle dorsiextensors, and muscles inserting on and originating from said spine and pelvis and the short and long ligaments adaptable to produce or involve with said disc herniation.

As a result, the measurements of lengths of a muscle or a plurality of muscles originating from or inserting on said spine and pelvis, the short and long ligaments, measurements angles formed by the long axis of a muscle or a plurality of muscles and the long axis of said spine, measurements of a torque/force or torques/forces exerted by a muscle or a plurality of groups of muscles on said spine and pelvis serve to fulfill the objectives of the present invention and in the spirit of the invention and the scope of the claims.

The present invention comprises a data means for predicting or estimating the risks, incidences and propensity towards said disc herniation in said individuals. The data means relates to disc herniation or herniations in said individuals comprises the characteristics and activities of individuals in a population who had disc herniation or herniations. The data means comprises, first, a ratio or a plurality of ratio's of male:female, a plurality of ranges, averages, medians and standard deviations of ages of males and females who sustained or had disc herniation or herniations. Therefore, said data means for predicting or estimating the risks, incidences and propensity towards said disc herniation is adaptable to identify other individuals in a population who are prone, susceptible to or at risk for disc herniation.

For example, the data indicates that there are more males who are at higher risk or are more prone or more susceptible to disc herniation than females. One ratio in said data is such as, but not limited to, male:females=approximately 2:1. The data means further comprises, invididuals experiencing disc herniation having a range of ages of approximately 30-61 years, average age of approximately 40 years, median age of approximately 38 years and standard deviations of about 9. Although said numbers merely represent one of the preferred embodiments of said data means of the present invention, it will be appreciated by those skilled in the art that variations of the values and parameters of said numbers in said ratio's, age ranges, averages or means, medians and standard deviations in males and females can and will be derived without departing from the spirit of the invention and the scope of the claims.

Similarly, said data means comprises data on the body types; measurements of lengths of a plurality of muscles originating from said spine; measurements of the lengths of a plurality of muscles inserting on said spine; measurements of lengths of a plurality of muscles originating from said pelvis; measurements of lengths of a plurality of muscles inserting on said pelvis; measurements of lengths of a plurality of muscles originating from the legs; measurements of lengths of a plurality of muscles inserting on the legs; measurements of a plurality of angles formed by the long axes of a plurality of muscles and the long axis of said spine; measurements of a pluraltiy of torques and forces exerted by a plurality of muscles on said spine; and types of physical activities and works. Without details, however, it will be appreciated by those skilled in the art that similar uses supra of said information will be had within the spirit of the invention and the scope of the claims.

The present invention provides a formula or a plurality of formulas for the treatments including the rehabilitation of the soft tissues producing or involving with said disc herniation. The treatments include the rehabilitation of the muscle, a plurality of muscles, ligament, a plurality of ligaments or a combination thereof that produces, produce, involves or involve with said disc herniation. For example, the rehabilitation includes elongation or stretching of a specific muscle, a plurality of specific muscles, ligament, a plurality of ligaments or any combination thereof. For example, the intervertebral muscles, hip flexors, hip extensors or any combination thereof are the candidates. Moreover, rehabilitation further includes the strengthening and conditioning of a specific muscle, a plurality of specific muscles, ligament, a plurality of ligaments or any combination thereof. Collorary, the rehabilitation includes the relaxation of a specific muscle, a plurality of specific muscles, ligament, a plurality of ligaments or any combination thereof such as, but not limited to, the hip flexors.

Although the preferred embodiments of the present invention have been described, it will be appreciated by those skilled in the art that adaptations and variations of the methods and means may be made without departing from the spirit of the invention or the scope of the claims.

1. A method of preventing intervertebral disc herniation and related disorders in a human or an animal spine comprising, first, a discovery that torques, forces, stresses, strains, sprains imposed on said spine, the pelvis and the spinal and pelvic components and the deviations in the musculoskeletal structure and biomechanics of said human or animal is a cause of said herniation of an intervertebral disc or a plurality of intervertebral discs and, second, a data means for estimating the risks, incidences and propensity towards said disc herniation in a human population or an animal population comprises:
an application of said discovery for the prevention of said disc herniation;
an application of said discovery for the treatment of of said disc herniation;
an illustration of said data;
an application of said data for the prevention of said disc herniation;
an application of said data for the treatment of of said disc herniation;
an identification of the soft tissues producing or involving with said disc herniation;
a treatment of the soft tissues producing or involving with said disc herniation;
a measurement of a length of a muscle originating from said spine;
a measurement of the lengths of a plurality of muscles originating from said spine;
a measurement of a length of a muscle inserting on said spine;
a measurement of the lengths of a plurality of muscles inserting on said spine;
a measurement of a length of a muscle originating from the pelvis;
a measurement of a length of a muscle inserting on the pelvis;
a measurement of the lengths of a plurality of muscles originating from the pelvis;
a measurement of the lengths of a plurality of muscles inserting on the pelvis;
a measurement of a length of a muscle originating from the leg;
a measurement of a length of a muscle inserting on the leg;
a measurement of the lengths of a plurality of muscles originating from the leg;
a measurement of the lengths of a plurality of muscles inserting on the leg;
a measurement of an angle formed by the long axis of a muscle and the long axis of said spine;
a measurement of a plurality of angles formed by the long axes of a plurality of muscles and the long axis of said spine;
a measurement of a torque exerted by a muscle on said spine;
a measurement measuring the torques exerted by a plurality of muscles on said spine;
a measurement of a force exerted by a muscle on said spine; and
a measurement of forces exerted by a plurality of muscles on said spine.

2. The method according to claim 1 wherein said deviations in the musculoskeletal structure and biomechanics according to claim 1 include the shortening and contracture of a specific muscle relating to said spine.

3. The method according to claim 1 wherein said deviations in the musculoskeletal structure and biomechanics according to claim 1 include the shortening and contracture of a plurality of specific muscles relating to said spine.

4. The plurality of muscles according to claim 3 include the shortening, spasm, contracture or a combination thereof of the hip flexors.

5. The plurality of muscles according to claim 3 include the shortening, spasm, contracture or a combination thereof of the hip extensors.

6. The method according to claim 1 wherein said illustration of said data means for estimating the risks, incidences and propensity towards said disc herniation comprises the characteristics and activities of individuals who had said disc herniation or herniations.

7. The data means according to claim 6 comprises a plurality of ratio's of male:female; age ranges, averages, means, medians and standard deviations in males and females; types of bodies; measurements of lengths of a plurality of muscles originating from said spine; measurements of the lengths of a plurality of muscles inserting on said spine; measurements of lengths of a plurality of muscles originating from said pelvis; measurements of lengths of a plurality of muscles inserting on said pelvis; measurements of lengths of a plurality of muscles originating from the legs; measurements of lengths of a plurality of muscles inserting on the legs; measurements of a plurality of angles formed by the long axes of a plurality of muscles and the long axis of said spine; measurements of a pluraltiy of torques and forces exerted by a plurality of muscles on said spine; and types of physical activities and works.

8. The method according to claim 1 wherein said identification of the soft tissues producing or involving with said disc herniation comprises the identification of specific muscles in the muscle groups of intervertebral muscles, abdominal and back muscles, hip flexors, hip extensors, ankle dorsiflexors and ankle dorsiextensors, and muscles inserting on and originating from said spine and pelvis adaptable to produce or involve with said disc herniation.

9. The method according to claim 1 wherein said identification of the soft tissues producing or involving with said disc herniation comprises the identification of specific short and long spinal ligaments that cause or involve with said disc herniation.

10. The method according to claim 1 wherein said a treatment of the soft tissues producing or involving with said disc herniation comprises a formula means for rehabilitating the muscles produce or involve with said disc herniation.

11. The method according to claim 1 wherein said a treatment of the soft tissues producing or involving with said disc herniation comprises a formula means for rehabilitating the ligaments produce or involve with said disc herniation.

12. The formula means for rehabilitating according to claim 10 includes a plurality of formulas for the elongation or stretching of a specific muscle.

13. The formula means for rehabilitating according to claim 10 includes a plurality of formulas for the elongation or stretching of a plurality of specific muscles.

14. The formula means for rehabilitating according to claim 13 includes a plurality of formulas for the elongation or stretching of the intervertebral muscles.

15. The formula means for rehabilitating according to claim 13 includes a plurality of formulas for the elongation or stretching of the hip flexors.

16. The formula means for rehabilitating according to claim 10 includes a plurality of formulas for the strengthening of a specific muscle.

17. The formula means for rehabilitating according to claim 10 includes a plurality of formulas for the strengthening of a plurality of specific muscles.

18. The formula means for rehabilitating according to claim 10 includes a plurality of formulas for the relaxation of a plurality of muscles.

19. A nonsurgical treatment of intervertebral disc herniation and related disorders in a human or an animal spine comprising, first, a discovery that torques, forces, stresses, strains, sprains imposed on said spine, the pelvis and the spinal and pelvic components and the deviations in the musculoskeletal structure and biomechanics of said human or animal is a cause of said herniation of an intervertebral disc or a plurality of intervertebral discs and, second, a data means for estimating the risks, incidences and propensity towards said disc herniation in a human population or an animal population comprises:
an illustration of said data;
an application of said discovery for the treatment of of said disc herniation;
an application of said data for the treatment of of said disc herniation;
an identification of the soft tissues producing or involving with said disc herniation;
a treatment of the soft tissues producing or involving with said disc herniation;
a measurement of a length of a muscle originating from said spine;
a measurement of the lengths of a plurality of muscles originating from said spine;
a measurement of a length of a muscle inserting on said spine;
a measurement of the lengths of a plurality of muscles inserting on said spine;
a measurement of a length of a muscle originating from the pelvis;
a measurement of a length of a muscle inserting on the pelvis;
a measurement of the lengths of a plurality of muscles originating from the pelvis;
a measurement of the lengths of a plurality of muscles inserting on the pelvis;
a measurement of a length of a muscle originating from the leg;
a measurement of a length of a muscle inserting on the leg;
a measurement of the lengths of a plurality of muscles originating from the leg;
a measurement of the lengths of a plurality of muscles inserting on the leg;
a measurement of an angle formed by the long axis of a muscle and the long axis of said spine;
a measurement of a plurality of angles formed by the long axes of a plurality of muscles and the long axis of said spine;
a measurement of a torque exerted by a muscle on said spine;
a measurement measuring the torques exerted by a plurality of muscles on said spine;
a measurement of a force exerted by a muscle on said spine; and
a measurement of forces exerted by a plurality of muscles on said spine.

20. A preventive medicine of disc herniation comprising a means for preventing and nonsurgically treating intervertebral disc herniation in humans and animals comprising a discovery that torques, forces, stresses, strains, sprains imposed on said spine, the pelvis, the spinal and pelvic components and the deviations in the musculoskeletal structure and biomechanics of said humans or animals and a data relating to the intervertebral disc herniation in individuals in a human population or an animal population wherein said data comprising the characteristics of individuals who had disc herniation or herniations adaptable, first, to elucidate said characteristics of individuals, second, serve as a predictor and pointer of the risks and incidences of said disc herniation and as an identifier of individuals who are prone or susceptible to said herniation, and, third, to guide the nonsurgical treatment and management of said disc herniation in said individuals comprises:
an application of said discovery for the prevention of said disc herniation;
an application of said discovery to identify the individuals who are at risk for said disc herniation;
an application of said discovery to identify the individuals who are prone or susceptible to said disc herniation;
an application of said discovery for the treatment and management of said disc herniation;
an application of said data for the prevention of said disc herniation;
an application of said data to identify the individuals who are at risk for said disc herniation;
an application of said data to identify the individuals who are prone or susceptible to said disc herniation;
an application of said data for the treatment and management of said disc herniation;
a formula means for rehabilitating the muscles produce or involve with said disc herniation; and
a formula means for rehabilitating the ligaments produce or involve with said disc herniation.
